(19) United States
(12) Patent Application Publication
(10) Pub. No.: US 2008/0076170 A1
(43) Pub. Date: Mar. 27, 2008

(54) Cell culture insert and cell culture vessel

(76) Inventors: Tuija Annala, Siivikkala (FI); Susanna Miettinen, Siivikkala (FI); Susanna Narkilahti, Tampere (FI)

Correspondence Address: VENABLE LLP, P.O. BOX 34385, WASHINGTON, DC, 20043-9998, US

(21) Appl. No.: 11/527,404

(22) Filed: Sep. 27, 2006

(51) Int. Cl. C12M 1/12; C12M 3/00

(52) U.S. Cl. 435/297.4; 435/305.1; 435/305.2

(57) ABSTRACT

A cell culture insert including a body having a grip extending sideways from the body, and a locking ring adapted to be fitted around the body. The grip is able to retract elastically inwards so that the body can be inserted inside a cell culture vessel with its grip in friction contact against interior sidewalls of a cavity of the cell culture vessel. The cell culture insert is kept in place by the friction contact and its height position can be adjusted inside the cavity.

### FIELD OF THE INVENTION

This invention relates to an apparatus for growing cells or tissue culture in vitro. More particularly, the invention relates to a new and improved cell culture insert for supporting tissue cultures in a fluid medium containing nutrients which promote the tissue culture growth. In another aspect the invention relates to a new and improved cell culture vessel.

### BACKGROUND OF THE INVENTION

Different kind of cell culture inserts and devices are described in European patent nos 0590513 B1, 0495213 B1, U.S. Pat. Nos. 5,026,649, and 5,116,496, US patent application publication nos 2006/0051857 A1 and US 2005/0112030 A1, and International patent application no WO 91/06368.

European patent no 0590513 B1 describes cell culture insert made of plastic material with a membrane on the bottom surface thereof so that there is free diffusion and transport of ions and macromolecules. In this invention membrane is fixed permanently in the insert, it is not possible to place the insert into the very bottom of the tissue culture vessel, and the height of the insert from the bottom of the tissue culture vessel is fixed. Corners of the insert are providing centering of the insert, they are not fixing the insert in the tissue culture vessel, but flanges on the top of the insert are laying on the top of the tissue culture vessel and providing support thereof.

European patent no 0495213 B1 discloses a culture cell which comprises cell culture insert with microporous membrane as the substrate for the cells being cultured. Even more particularly, this invention relates to such a cell culture insert having an arrangement for breaking away the bottom supporting portion of the membrane, once the cells have been properly developed, as required, so as to remove the supporting structure of the insert to eliminate its presence during subsequent examination of the cells having been developed in the insert. It is not possible to place the insert into the very bottom of the tissue culture vessel and the height of the insert from the bottom of the tissue culture vessel is fixed. The flanges on the top of the insert are laying on the top of the tissue culture vessel and providing support thereof.

US patent application publication no US2006/0051857 A1 introduces a cell culture insert comprising a beaker-shaped insert wall, a base consisting of a membrane filter, projecting support arms that are distributed around the periphery of the top and lateral spacers. According to the invention, the spacers are distributed around the periphery of the cell culture insert and have different radial lengths in such a way that a large feed opening and several smaller openings are formed

U.S. Pat. No. 5,026,649 describes an apparatus for growing tissue cultures in vitro, which permits a concentration gradient of nutrients to develop through a permeable membrane to which a sample of tissue is attached. The permeable membrane is attached to the bottom end of tubular support that in turn hangs by a flange connected to its upper end on the top of a well containing the nutrients. The upper end of the tubular support includes an outwardly extending flange which in turn carries a rim that can hang upon the upper end of a well in a tissue culture cluster dish. The flange and rim together form a shoulder that precisely positions and centers the support in the well and prevents it from shifting laterally in the well.

US patent application publication no US 2005/0112030 A1 discloses a versatile Meshwell plate. This invention enables simultaneous rapid and high-throughput processing of small tissue samples or organisms. In an embodiment, the Meshwell plate consists of 96 meshwells and is particularly useful in assaying zebrafish embryos. The bottom tips of standard 96-well PCR plates are removed and replaced by a mesh with openings of about 75-300 μm, preferable 150 μm, in size.

US patent no U.S. Pat. No. 5,116,496 introduces a well for use in microtitration and/or microfiltration procedures. The well has a membrane in the bottom that is retained on a lower lip by means of a ring that is itself retained at predetermined position in, and along the wall of, the well.

International patent application no WO 91/06368 discloses an invention, which comprises a hollow tubular member with open ends, the internal and/or external surface of which is shaped as a zig-zag or pleated wall to provide an extended surface area for a reagent coated on the insert wall surface.

The solutions of prior art fail to provide a cell culture insert which can be used in a variety of ways for keeping a cell culture substrate at a desired position inside a cavity of a cell culture vessel, either in contact with the bottom of the cavity, close to the bottom or at a height that can be chosen independently of the dimensions of the cell culture insert. Furthermore, cell culture vessels containing cell culture inserts of prior art suffer largely from the disadvantages connected with the construction of these inserts when used for the culture of cells.

### SUMMARY OF THE INVENTION

It is an object of the invention to provide a cell culture insert that eliminates the above-mentioned drawbacks. It is a further object of the invention to provide a cell culture insert that can be fixed in a cell culture vessel despite minor dimensional differences between culture vessels of standard size cavities that are due to different manufacturers.

According to the present invention, the cell culture insert comprises two principal parts, the body and the ring. The insert can be preloaded with film, foil, fibre, textile, membrane, mesh or tissue sample, or the customer may load it with the desired material by themselves. The insert can be fitted against the bottom of a cavity of the tissue culture vessel or raised to the required height from the bottom. The insert acts as a sort of holder of a cell culture substrate or tissue sample inside the cell culture vessel and it has been made of medical grade plastic or comparable material, which does not transmit any foreign substances harmful to cell culture.

The cell culture insert according to the present invention comprises a plastic material body and ring. The substrate in the form of film, foil, fibre, textile, membrane, mesh, or a self-supporting tissue sample will be cut so that its diameter is bigger than that of the body. Thereafter, the sample will be set on the ring and the body is pushed to its place. Now the insert with the sample is ready to be placed into the tissue culture vessel, for example a well of a well plate. After the necessary period of cell culture, the insert will be removed from the tissue culture vessel, the ring is removed and thus, the cell transplant with viable cells is ready.

The cell culture insert may be formed in different sizes and geometric configurations so as to be used with different sizes and geometric configured tissue culture vessels. However, by virtue of the elastically yielding gripping means which may take many shapes, the body of a cell culture insert designed for a vessel that has a cavity of a certain standard inner diameter can be fitted inside the vessel and secured at any height position despite of minor differences in the diameters of these “standard” vessels.

Preferably, the cell culture insert comprises three to six wings along the sides of the body for fulfilling the function of gripping means. The wings provide means for self centering the cell culture insert when positioned for use in the cell culture vessel that may have a well of a circular geometric configuration. Furthermore, a pipette may be inserted between side walls of the cell culture insert and the well of the tissue culture vessel. The pipette may be inserted without disturbing or removing the cell culture insert from the well to reach the bottom of the well of the tissue culture vessel and introduce or remove medium from beneath of the cell culture insert.

The exterior dimensions of the portion of the cell culture insert between the gripping means are sufficiently less than the interior diameter of the cavity of the cell culture vessel to allow a pipette or similar device to be positioned between the walls for fluid filling or aspiration.

The wings or equivalent gripping means allow free adjustment of height of the insert from the bottom of the well of the tissue culture vessel. The wings may also provide an area for gas exchange.

An important feature of the cell culture insert of the present invention is the means to remove the ring from the body after the desired cultivation period and unload the substrate with cells or the self-supporting tissue sample without disturbing the viability of the cells. Then the sample is ready for installation in vivo in human or animal or testing of effect of different pharmaceuticals or other ingredients in vitro.

The insert according to the invention can also be utilized to keep a hard sample in its place at the bottom of the tissue culture vessel. In that case, the sample will be cut slightly smaller in size than the opening of the tissue culture vessel. The sample is placed on the bottom of the tissue culture vessel and the insert is pushed on top of it. For this purpose, it is possible to use the body or ring alone or the ensemble of the body and the ring.

This invention provides the option to use the insert with variety of materials and samples chosen by the researchers. Since the insert can be positioned down in the cavity of a cell culture vessel so that no parts of it project above the upper edge of the cavity, a lid or plug can be used for closing the upper opening of the cavity unobstructed by the insert.

### BRIEF DESCRIPTION OF THE DRAWINGS

Further objects, features and advantages of the present invention will be more fully appreciated by reference to the following detailed description of the presently preferred but nonetheless illustrative embodiments in accordance with the present invention when taken in conjunction with the accompanying drawings wherein

FIG. 1 illustrates the parts of the cell culture insert and their assembly in vertical section;

FIG. 2 illustrates the introduction of the insert in a tissue culture vessel in vertical section;

FIG. 3 shows an alternative embodiment of a cell culture insert placed in a culture vessel in vertical section;

FIG. 4 illustrates different possible height positions of the cell culture insert in a culture vessel in vertical section;

FIG. 5 is a perspective view of the parts of the cell culture insert;

FIG. 6 is a perspective view of the parts of the cell culture insert according to another embodiment; and

FIG. 7 is a perspective view of the parts of the cell culture insert according to one further embodiment.

### DETAILED DESCRIPTION OF THE PREFERRED EMBODIMENTS OF THE INVENTION

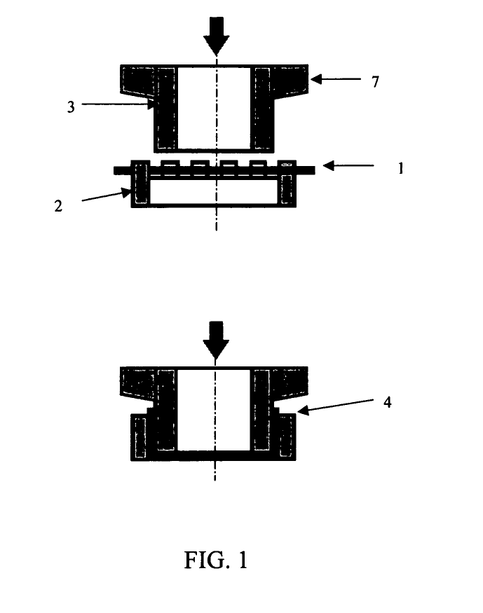

Installation of the body, cell growth material and ring is represented in FIG. 1. The desired cell growth material 1, which can be a substrate for cells, such as film, foil, fibre, textile, membrane, mesh, or consist of cells, such as a self-supported tissue sample is placed on the ring 2, the body 3 is pushed into its place so that the ring 2 fits tightly enough around the outer wall of the body 3 at the lower part of the body in such a manner that it is in sliding engagement with the body with the margin areas of the material 1 between the inner surface of the ring 2 and the outer surface of the body 3. In this way the material 1 is secured on the body 3 so that it covers the lower opening of the body. The cell culture insert 4 is now ready for placement into the desired tissue culture vessel 5. The body has the general shape of a hollow cylindrical sleeve, having typically greater height than wall thickness that the sleeve will be positioned in vertical position inside the cell culture vessel. The horizontal cross-section of the sleeve is substantially circular, which is advantageous for inspection of the cells of the material 1, for example by microscope.

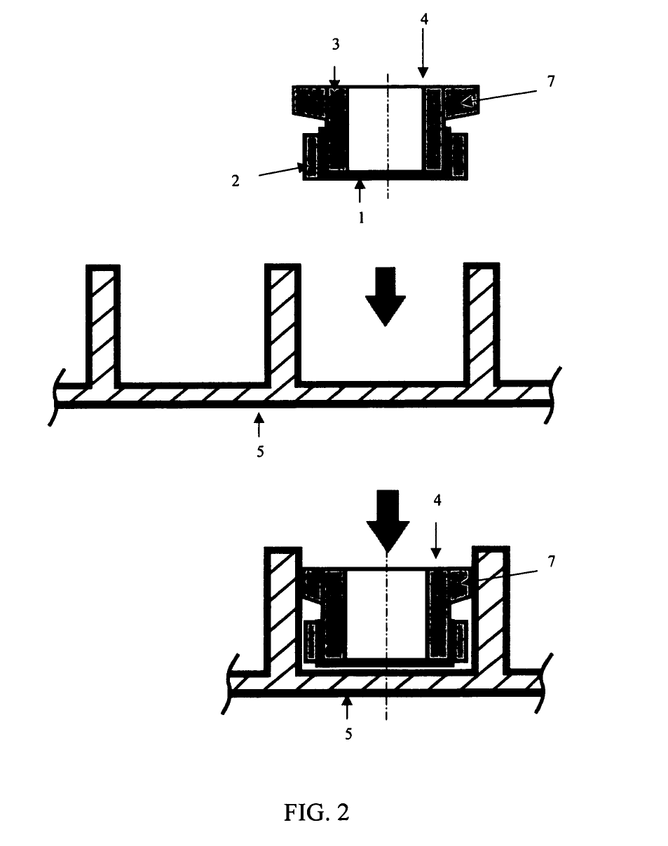

FIG. 2 shows the insertion of the sleeve-like body 3 together with the ring 2 and material 1 inside a cell culture vessel. The body has gripping means 7 extending sideways from the outer wall of the body 1. The gripping means 7 are able to retract elastically inwards so that the body can be inserted inside a cavity the cell culture vessel with its gripping means in friction contact against interior sidewalls of the cell culture vessel and kept in place by means of said friction contact. FIG. 2 further shows that the ring can be placed around the body so that it will lie entirely above the lower end of the body. The material 1 will thus be lowest in the assembled cell culture insert 4 and the material itself can thus be brought as close to the bottom of the cavity as possible, even in direct contact with the bottom if desired.

The apparatus for growing tissue cultures as shown in FIG. 2 includes a cell culture vessel 5 and the cell culture insert 4. Although only two wells in the cell culture vessel is shown, it should be appreciated that the cell culture vessel may have one, six, eight, twelve, twenty-four or some other number of wells, i.e. cavities, selected for the particular purpose for which the apparatus is used. The cell culture vessel can be a well-plate, also called multiwell plate, having a matrix of n×m wells in the fashion known per se. The wings are flexible and decrease their opening (extension sideways from the body) upon installation.

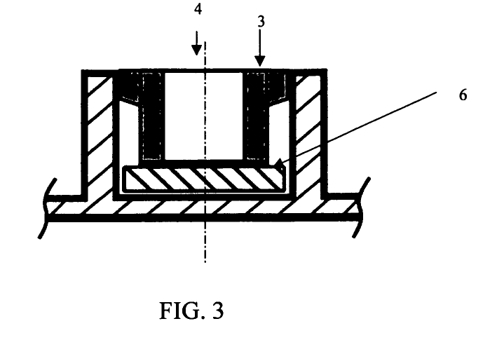

In FIG. 3 the body 3 is used to keep a hard block sample 6 in its place at the bottom of the tissue culture vessel (cavity of the vessel). In that case, the sample will be cut slightly smaller in size than the opening of the cavity of the tissue culture vessel 5. The sample 6 is placed on the bottom of cavity and the insert is pushed on top of it. For this purpose, it is possible to use the body 3 alone or the ensemble of the body and the ring 4. The hard block sample 6 can be porous to allow the growth of cells inside it. Although shown slightly above the bottom in FIG. 3, it will be appreciated that the sample 6 can be pushed down against the bottom of the vessel by the lower end of the body 3.

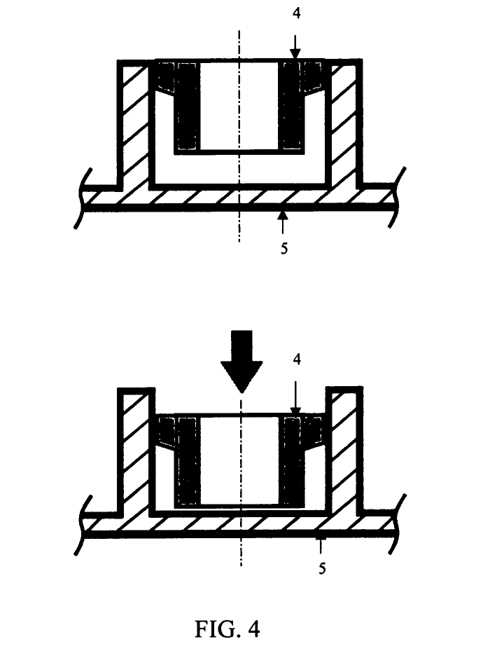

FIG. 4 illustrates the possibility to control the height of the cell culture insert 4 in the well, the material 1 and the ring 2 being omitted. Depending on the needs, the insert can be placed on the bottom of the well or heighten it up to the level of the upper surface of the tissue culture vessel 5. The adjustment of the height position is stepless, because the insert can slide with the gripping means 7 in friction contact with the inner wall of the well. The height of the insert 4 can thus be selected so that the cell growth material 1 fixed to it lies at optimum height for the purpose of cultivation of cells. If the assembly of the ring 2 and the sleeve-like body 3 has a total height lower that the inner height of the cavity (well) of the cell culture vessel 5, it can be placed totally inside the cavity so that a lid or plug can be used for closing the upper opening of the cavity, the closing means being thus able to be in contact with the upper edge of the cavity unobstructed by the cell culture insert 4.

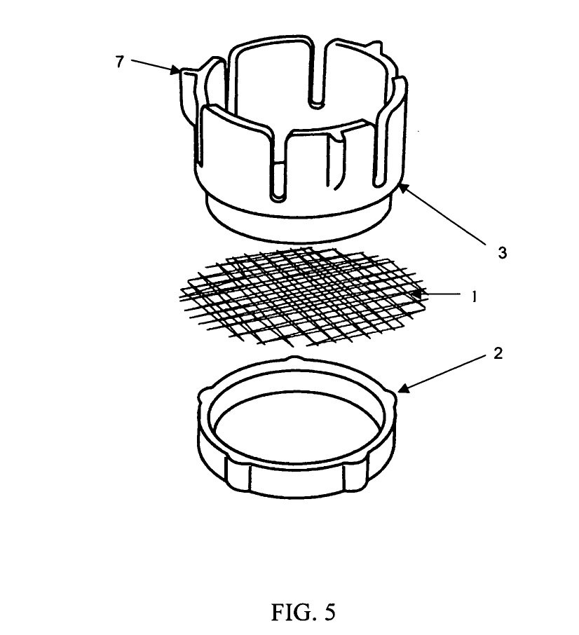

FIGS. 5-7 show different designs of the cell culture insert.

FIG. 5 shows an insert 4 where the body 3 has a plurality of gripping means 7 distributed at even intervals around the outer periphery of the body 3. The gripping means 7 are in form of vertical ridges protruding from the outer wall of the sleeve. Their elasticity inwards is ensured by cutout portions or notches extending from the upper edge of the sleeve and terminating midways of the sleeve. The cutout portions or notches limit tongues that comprise said ridges and that can slightly bend inwards to allow the elastic movement of the gripping means. The lower part of the sleeve has a smaller diameter so that the ring 2 can be fitted around the body 3 in position, where the ring 2 does not extend radially beyond the gripping means 7 in horizontal direction.

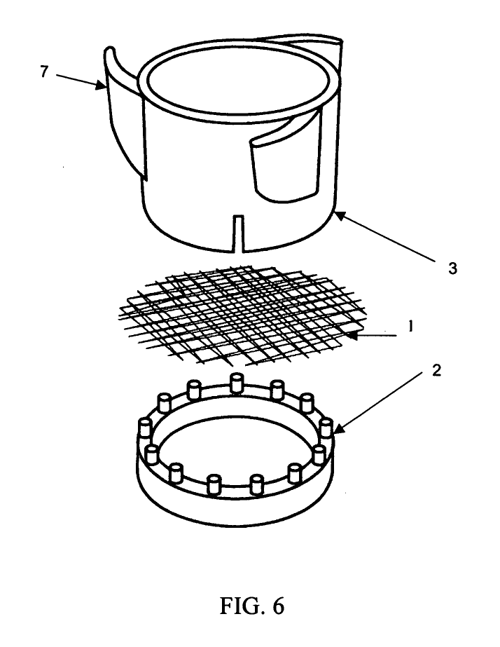

FIG. 6 shows an insert 4 where the gripping means 7 are in the form of elastic wings that extend obliquely from the relatively rigid outer wall of the sleeve and are slightly curved. The wings are able to bend inwards so that they can fulfil the function of elastic gripping means. The ring 2 has on its upper surface short pins or knobs that help to keep the material 1 in place when the body is pushed down into the ring 2. In this embodiment of the insert, the ring 2 also has a smaller outer diameter that the outermost diameter of the body 3 defined by the wings. At the lower part of the body 3 there are cutout portions or notches starting from the lower end of the sleeve to increase the elasticity of the sleeve in this zone so that the ring 2 could be more easily placed around it.

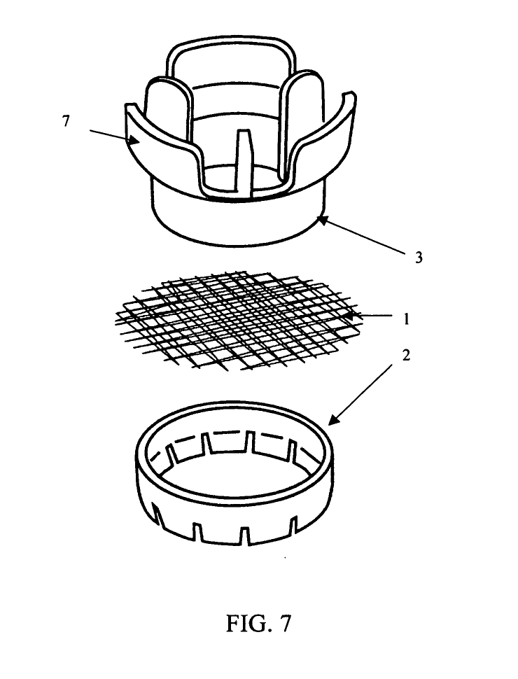

FIG. 7 in turn shows an insert 4 where the gripping means 7 are formed of elastic arcuate portions of the sleeve wall that are left between cutout portions or notches in the sleeve wall and diverge slightly conically in upward direction. Radially extending wings situated centrally of the cutout portions or notches serve to stiffen the body 3. The lower part of the body 3 has a smaller diameter and the ring 2 has cutout portions to facilitate its installation around the lower part of the body 3.

The culture vessel where the insert is located includes a base comprising a number of wells each comprising an inner side wall closed at the bottom by wall and open at the top end. The base is typically transparent and may be, for example, molded of polyvinylchloride or polypropylene. For viewing of the cell culture by microscope it is important that at least the bottom of the cavity is transparent.

It can be seen that the body 3 of the cell culture insert 4 has a varying outer diameter which is largest in the area of the gripping means 7. This will leave areas between the gripping means for adding or withdrawing substances between the body and the inner wall of the cavity into or from the cavity.

While in the foregoing paragraphs the details of the culture vessel illustrated are described, it is to be appreciated that the vessel itself does not form part of the present invention, and the cell culture insert of the present invention may be sized to fit and be used with other culture vessels or like receptacles. However, the combination of a cell culture insert and the vessel, containing also the material for cell growth, forms part of the present invention. According to one embodiment, a vessel having a plurality of cavities, of which at least some or all are provided with a cell culture insert together with a cell culture substrate can be provided ready for the customer.

The cell culture insert can be delivered to the customer without a cell culture substrate, which can be selected by the customer when using the insert for cell culture purposes. Alternatively, the cell culture substrate can be pre-assembled in the insert which is thus delivered together with the substrate to the customer.

As practitioners-in-the-art will understand, the cell inserts of the invention may be comprised of simple moldable parts which may be mass produced from a variety of materials, including, for example, polyethylene, polystyrene, polyethylene terephthalate, and polypropylene.

As to the material for the cell culture substrate, it can be made of synthetic and natural biomaterials including but not limited to polylactides, polyglycolides, poly-d,l-lactides, polyethylene oxides, polyepsilon caprolactones, chitosan, collagen, starch, bioglass, hyaluronic acid, tyrosine, tricalciumphosphate, hydroxyapathite, polyurethane, alginate and fibrin, and polypropylene and their copolymers, including mixtures of the above-listed materials. The cell culture substrate can be in the form of foil, fiber, textile, membrane, mesh, block, foam, or plate. Especially in case of blocks held on the bottom of the culture vessel, they can be of tricalciumphosphate or hydroxyapathite. The block can also be made of fibers or foam which form a porous felt-like or sponge-like porous structure. As can be seen from the above listing, the materials can be biodegradable or stable.

The material in connection with the insert can be tissue itself of human or animal origin, such as amniotic membrane, bone, soft tissue or cornea. The membrane can be used in the same way as the material 1 in FIGS. 1, 2 and 5-7, i.e it spans the lower opening of the sleeve. The bone, soft tissue and cornea can be used in the same way as the block in FIG. 3, that is, held against the bottom by the sleeve.

What is claimed is:

1. A cell culture insert, comprising
a body having gripping means extending sideways from the body
a locking ring adapted to be fitted around the body;
wherein said gripping means are able to retract elastically inwards so that the body can be inserted inside a cell culture vessel with its gripping means in friction contact against interior sidewalls of a cavity of the cell culture vessel and kept in place by means of said friction contact.

2. The cell culture insert according to claim 1 wherein the cell culture insert further comprises a cell culture substrate fixed by means of the ring to the body.

3. The cell culture insert according to claim 2 wherein the cell culture substrate is foil, fibre, textile, membrane, or mesh.

4. A cell culture vessel, comprising
a cell culture insert inside a cavity of the cell culture vessel, said cell culture insert comprising gripping means in friction contact against interior sidewalls of the cavity of the cell culture vessel to keep the cell culture insert in place by means of said friction contact; and
a cell culture substrate or tissue sample retained inside said cavity of the cell culture vessel by said cell culture insert.

5. The cell culture vessel according to claim 4, wherein said cell culture substrate or tissue sample is locked to the body of the cell culture insert by means of a ring fitted around the body.

6. The cell culture vessel according to claim 4, wherein said cell culture substrate or tissue sample is placed in the cavity of the cell culture vessel below the cell culture insert, which limits the upward movement of the cell culture substrate or tissue sample.

7. The cell culture vessel according to claim 4, wherein the cell culture vessel comprises a plurality of cavities, of which at least some contain the cell culture insert and the cell culture substrate or tissue sample.

8. The cell culture vessel according to claim 5, wherein the cell culture vessel comprises a plurality of cavities, of which at least some contain the cell culture insert and the cell culture substrate or tissue sample.

9. The cell culture vessel according to claim 6, wherein the cell culture vessel comprises a plurality of cavities, of which at least some contain the cell culture insert and the cell culture substrate or tissue sample.
